(19) United States
(12) Patent Application Publication
(10) Pub. No.: US 2001/0016732 A1
(43) Pub. Date: Aug. 23, 2001

(54) DUAL MODE LASER DELIVERY SYSTEM PROVIDING CONTROLLABLE DEPTH OF TISSUE ABLATION AND CORRESPONDING CONTROLLABLE DEPTH OF COAGULATION

(76) Inventors: JAMES L. HOBART, LOS ALTOS HILLS, CA (US); DANIEL K. NEGUS, LOS ALTOS, CA (US); DAN E. ANDERSEN, MENLO PARK, CA (US)

Correspondence Address: THOMAS B HAVERSTOCK, HAVERSTOCK & OWENS, 260 SHERIDAN AVENUE, SUITE 420, PALO ALTO, CA, 94306

(21) Appl. No.: 09/018,104

(22) Filed: Feb. 3, 1998

(51) Int. Cl. A61B 18/20

(52) U.S. Cl. 606/2; 606/9

(57) ABSTRACT

A dual mode laser delivery system provides a controllable depth of both ablation and coagulation of an area of skin to be treated. The laser delivery system preferably includes a laser source having a short penetration depth. The controllable ablation depth is achieved by providing an appropriate series of pulses from the laser having an energy and exposure time to achieve ablation of the exposed area of skin to the desired depth. Once ablation of the skin has been performed, a coagulation region to the desired coagulation depth is then generated within the remaining exposed layer of skin by preferably applying a series of one or more very short non-ablative laser pulses at a high repetition rate in order to raise the temperature of the surface of the skin to a desired temperature for a period of time. This series of coagulation pulses will also serve to raise the temperature of the skin under the surface of the skin to a temperature high enough to cause coagulation to the desired depth. The order of delivery of the ablation sequence and the coagulation sequence can also be reversed from that described if desired. A graphical user interface is included within the system in order to allow the user to easily select and monitor the necessary parameters such as ablation depth, coagulation depth, application order, scan pattern, scan size and rate of laser pulses. The laser pulses are generated from a laser source and delivered through an articulated arm. The articulated arm includes a series of relay focussing lenses in order to periodically refocus the laser beam as it travels through the articulated arm.

### FIELD OF THE INVENTION

The present invention relates to the field of medical lasers. More particularly, the present invention relates to the field of medical lasers for effecting tissue ablation and coagulation.

### BACKGROUND OF THE INVENTION

Lasers are used in medical procedures to rejuvenate, restore and resurface skin damaged due to many causes including prolonged exposure to the sun and wrinkling. As is well known, prolonged exposure to the sun causes damage to the skin's surface and to the layers of skin below the surface. The principle cause of this damage is believed to result from the depletion of the collagen layer. In medical procedures using lasers, laser energy is delivered to the surface of the skin in a controlled pattern in order to ablate or bum away layers of the skin. A zone of thermal necrosis is created within the newly exposed layer of skin. The thickness of this zone of thermal necrosis will depend at least in part upon the absorption length of the laser being used. As the layers of skin grow back within the area of skin exposed to the laser, the damaged layers are restored to an undamaged condition in order to effectively resurface the skin.

A carbon-dioxide (CO₂) laser has been used in such skin resurfacing procedures. The carbon-dioxide laser is a powerful laser. In skin or human tissue, the carbon-dioxide laser has a long penetration depth or absorption length. Application of a carbon-dioxide laser to an exposed area of skin can result in an ablation of skin at the spot to which the laser is delivered. Because of its long penetration depth in human tissue, use of a carbon-dioxide laser on skin will result in a formation of a coagulation region or zone of thermal necrosis within the remaining layers.

An exemplary crater within a treated area of human skin created by a carbon-dioxide laser is illustrated in FIG. 1. The crater 12 is created by delivery of a carbon-dioxide laser to the skin 10 for a predetermined period of time at a predetermined fluence, or energy level. Exposure of the skin to the carbon-dioxide laser will ablate the skin within the area or spot exposed to the laser down to a depth determined by the energy of the laser used and the time that the spot is exposed to the laser, thereby creating the crater 12. Exposure of this area of skin to the carbon-dioxide laser will also create the coagulation zone 14 below the now exposed top layer of skin within the crater 12. Typically, with a conventional carbon-dioxide laser, the coagulation zone 14 will have an exemplary thickness of 50 microns below the remaining top exposed layer of skin. This coagulation zone 14 is also referred to as a thermal necrosis layer or thermal damage layer. This is considered a deep thermal necrosis layer in that it is typically thicker than the capillary region.

Exposure to a carbon-dioxide laser will initially result in tissue which has a harsh appearance, the skin will look bruised or damaged. Over time this harsh appearance will lessen and eventually, the patient's skin will obtain a restored and resurfaced appearance. Carbon-dioxide lasers are generally used in the treatment and resurfacing of heavily damaged skin due to such things as long term exposure to the sun. These lasers find application in such treatments, because they have the ability to remove the damaged layer of skin and create a thick coagulation zone both of which are thought to be necessary in order to achieve a restoration or resurfacing of the treated skin. The coagulation or thermal necrosis region achieved with a carbon-dioxide laser allows this medical treatment to be used to rejuvenate greater depths of skin than other lasers due to the intraoperative hemostasis or stopping of blood flow through the exposed skin, resulting from the relatively thick coagulation zone created by exposure to this laser.

Erbium lasers are also used in medical procedures for skin resurfacing and the like. The erbium laser oscillates at a wavelength which is much more strongly absorbed in tissue than the carbon dioxide laser. Previous erbium lasers are also less powerful than carbon-dioxide lasers. The higher absorption coefficient coupled with the lower power results in a shorter depth of penetration in human skin for erbium than carbon-dioxide lasers. Because of this shorter depth of penetration, the erbium laser will create a thermal necrosis region which is much thinner than the thermal necrosis region created by a carbon-dioxide laser. Due to the shorter depth of penetration, the thermal necrosis region created by conventional application of an erbium laser will also not achieve a thick coagulation region and intraoperative hemostasis. Therefore, use of an erbium laser to ablate skin tissue to a certain depth usually results in bleeding from the treated area. In conventional applications, the depth of skin which can be ablated by pulses from an erbium laser is limited due to the failure of such pulses to achieve intraoperative hemostasis, since bleeding from the superficial dermal vessel plexus not only obscures the operating field, but effectively prevents further ablation due to the total absorption of the laser light in the thin layer of blood. Multiple pulses from an erbium laser have been used to ablate skin to a desired depth, but this technique is limited by the lack of intraoperative hemostasis achieved with an erbium laser.

An exemplary crater within a treated area of human skin created by an erbium laser is illustrated in FIG. 2. The crater 22 is created by delivery of an erbium laser to the skin 20 for a predetermined period of time at a predetermined fluence. Exposure of the skin to the erbium laser will ablate the skin within the area exposed to the laser down to a depth determined by the energy of the laser used and the time of exposure of the spot to the laser, creating the crater 22. Exposure of this spot of skin to the erbium laser will also create the thermal necrosis region 24 below the now exposed top layer of skin within the crater 22. With conventional application of an erbium laser, the thermal necrosis region 24 will have an exemplary typical thickness of 10 microns below the remaining top exposed layer of skin. This is considered a short thermal necrosis layer in that it is typically much thinner than the capillary region. Each pulse applied from an erbium laser will ablate a certain depth of skin and will result in a thermal necrosis region of this thickness.

Exposure to an erbium laser will also initially result in a harsh appearance. However, this appearance is not as harsh as the appearance created by the deeper wounding carbon-dioxide laser and improves in a much faster time period. Erbium lasers are generally used in the treatment and resurfacing of skin to remove skin blemishes such as superficial wrinkles.

Presently, a doctor or medical facility using lasers in medical procedures to resurface skin due to both prolonged exposure to the sun (deep rythids) and blemishes such as more superficial wrinkles, must have two medical lasers, including a carbon-dioxide laser and an erbium laser. The carbon-dioxide laser is used to treat patients desiring resurfacing of skin which is damaged due to prolonged exposure to the sun for which a thick coagulation zone is necessary. The erbium laser is used to treat patients desiring resurfacing of skin to remove blemishes such as wrinkles in which a thinner coagulation zone is acceptable. What is needed is a single laser which can be used in the resurfacing of skin which has been damaged due to both prolonged exposure to the sun and blemishes such as wrinkles. What is further needed is a single laser which can be used to provide a coagulation zone of a controllable depth ranging from the depth normally achieved with an erbium laser to at least the depth achieved using a carbon-dioxide laser, allowing the clinician to tune the depth of coagulation as appropriate for the type of tissue damage being corrected. What is still further needed is a single laser providing both a controllable ablation depth and a controllable coagulation depth.

### SUMMARY OF THE INVENTION

A dual mode laser delivery system provides a controllable depth of both ablation and coagulation of an area of skin to be treated. The laser delivery system preferably includes a laser source having a short penetration depth. The controllable ablation depth is achieved by providing an appropriate series of pulses from the laser having an energy and exposure time to achieve ablation of the exposed area of skin to the desired depth. Once ablation of the skin has been performed, a coagulation region to the desired coagulation depth is then generated within the remaining exposed layer of skin by preferably applying a series of one or more very short non-ablative laser pulses at a high repetition rate in order to raise the temperature of the surface of the skin to a desired temperature for a period of time. This series of coagulation pulses will also serve to raise the temperature of the skin under the surface of the skin to a temperature high enough to cause coagulation to the desired depth. The order of delivery of the ablation sequence and the coagulation sequence can also be reversed from that described if desired. A graphical user interface is included within the system in order to allow the user to easily select and monitor the necessary parameters such as ablation depth, coagulation depth, application order, scan pattern, scan size and rate of laser pulses. The laser pulses are generated from a laser source and delivered through an articulated arm. The articulated arm includes a series of relay focussing lenses in order to periodically refocus the laser beam as it travels through the articulated arm.

### BRIEF DESCRIPTION OF THE DRAWINGS

FIG. 1 illustrates an exemplary crater within a treated area of human skin created by a carbon-dioxide laser.

FIG. 2 illustrates an exemplary crater within a treated area of human skin created by an erbium laser.

FIG. 3 illustrates the laser system of the preferred embodiment of the present invention.

FIG. 4 illustrates a block diagram of the electrical components and connections within the laser system of the preferred embodiment of the present invention.

FIG. 5 illustrates a graphical user interface of the preferred embodiment of the present invention in a scanning mode.

FIG. 6 illustrates the graphical user interface of the present invention in a single shot mode.

FIG. 7 illustrates an enlarged example of the left side of the graphical user interface of the present invention.

FIG. 8 illustrates a graph showing the effect of the coagulation pulses of the present invention over time to depths within the skin area being treated.

FIG. 9 illustrates an ablation pulse delivered from the laser system of the present invention.

FIG. 10 illustrates a coagulation pulse sequence delivered from the laser system of the present invention.

FIG. 11 illustrates a single extended coagulation pulse.

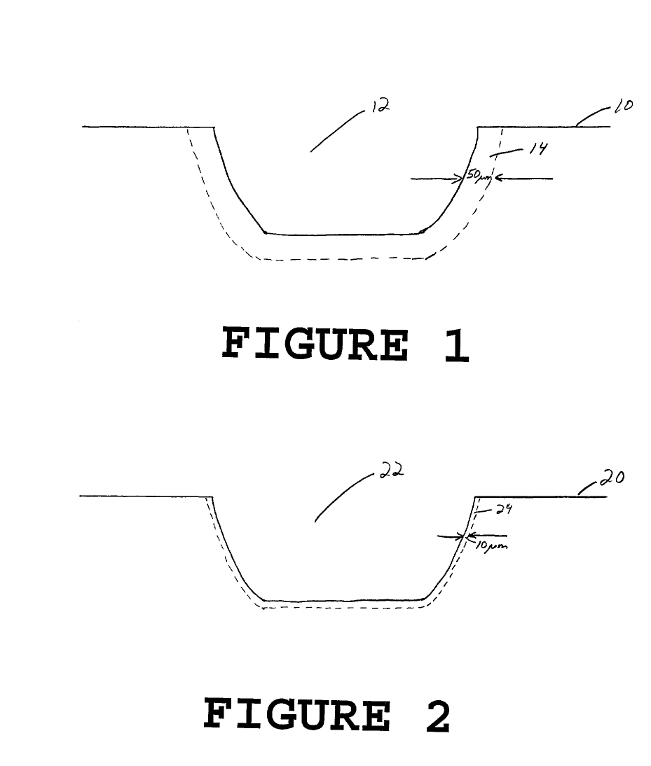

### DETAILED DESCRIPTION OF THE PREFERRED EMBODIMENT

A laser source having a short penetration depth is used to achieve a controllable ablation depth and a controllable depth of the resulting thermal necrosis zone (coagulation depth) below the remaining layer of exposed skin. Preferably, this laser is an erbium laser but the invention is not limited to it. The controllable ablation depth is achieved by providing an appropriate series of pulses from the laser having an energy and exposure time to achieve ablation of the exposed skin to the desired depth. Once ablation of the skin has been performed, a coagulation region is then created within the remaining exposed layer of skin. As described above, an ablation pulse from an erbium laser will create a coagulation region having a thickness of approximately 10 microns. If a thicker coagulation region is desired, the laser system of the present invention uses the erbium laser to generate this coagulation region to the desired depth. This coagulation region is generated to the desired depth by applying laser energy in a manner to allow conduction heating from the surface of the skin, but in a manner to avoid additional ablation. In the preferred embodiment, a series of very short laser pulses at a high repetition rate which are not energetic enough to achieve ablation, but which will maintain the surface of the remaining exposed layer of skin at a temperature which allows heat flow into the skin, thereby raising the temperature of the skin below the surface and producing coagulation to the desired depth.

The laser system of the present invention operates in both an ablation mode and a coagulation mode to achieve the desired treatment of the skin. These modes are combined in a selectable series of pulses within the laser system of the present invention to achieve a combination of ablation of an area of skin to a desired ablation depth and coagulation of the area of skin to a desired coagulation depth. In the ablation first mode, a sequence of ablation pulses is first applied to the area of skin to ablate skin within the target area down to the desired ablation depth and then a series of coagulation pulses is applied to create a coagulation zone within the remaining exposed layer of skin down to the desired coagulation depth. In the coagulation first mode, a sequence of coagulation pulses is first applied to the target area until the desired necrosis depth is achieved and then a sequence of ablation pulses is applied to remove tissue to a (presumably shallower) desired depth.

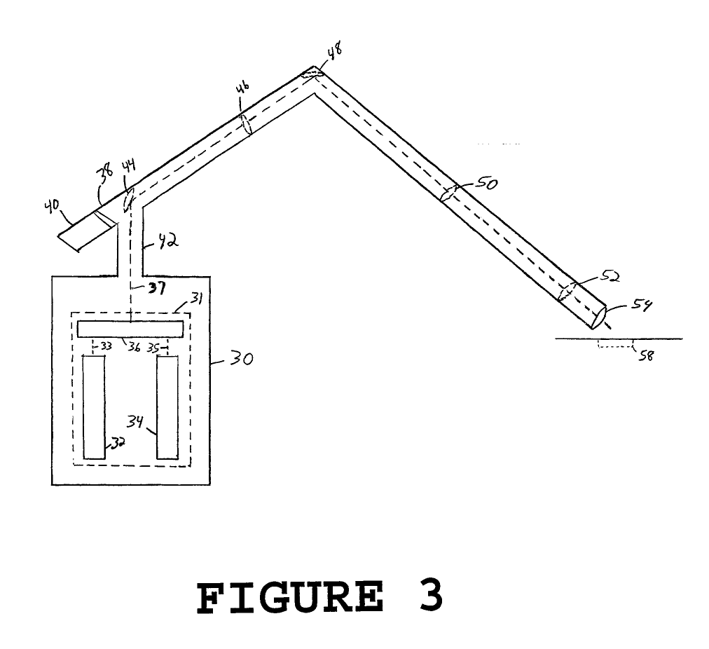

The laser system of the preferred embodiment of the present invention is schematically illustrated in FIG. 3. The laser generation system housing 30 includes the laser source 31 from which the laser beam 37 is provided. The laser source 31 preferably includes two erbium lasers 32 and 34 which generate the laser beams 33 and 35, respectively. Alternatively, any other appropriate short penetration length laser source can be used within the system of the present invention. The two laser beams 33 and 35 are combined into a single laser output 37 by the galvonometer 36 which switches between the two laser outputs 33 and 35. The galvonometer 36 then provides the laser output 37 from the laser source 31. An articulated delivery arm 38 is mounted onto the laser generation system housing 30 and directs the laser output 37 from the laser source 31 through the arm 38, to the scanner handpiece 54 where it is delivered to the area of skin 58 which is to be treated. The articulated arm 38 includes a weighted counterbalance 40 in order to reduce the mass necessary for the clinician to support during use. The laser output 37 is directed from the laser source 31 to a first series of directing optics 44, which are conventionally turning mirrors, to direct the laser output 37 through the arm 38 towards the joint of the arm. As will be described in further detail below, the articulated arm 38 also includes a number of focusing lenses for focusing the laser output 37 as it is directed through the arm 38. From the first directing series of lenses 44, the laser output 37 travels through the first focusing lens 46 to the second directing series of lenses 48 which direct the laser output 37 through the joint of the arm towards the scanner handpiece 54. From the second directing series of lenses 48, the laser output 37 travels through the focussing lenses 50 and 52. From the focussing lens 52, the laser output 37 travels through the scanner handpiece 54 and is provided to the area of skin 58 to be treated.

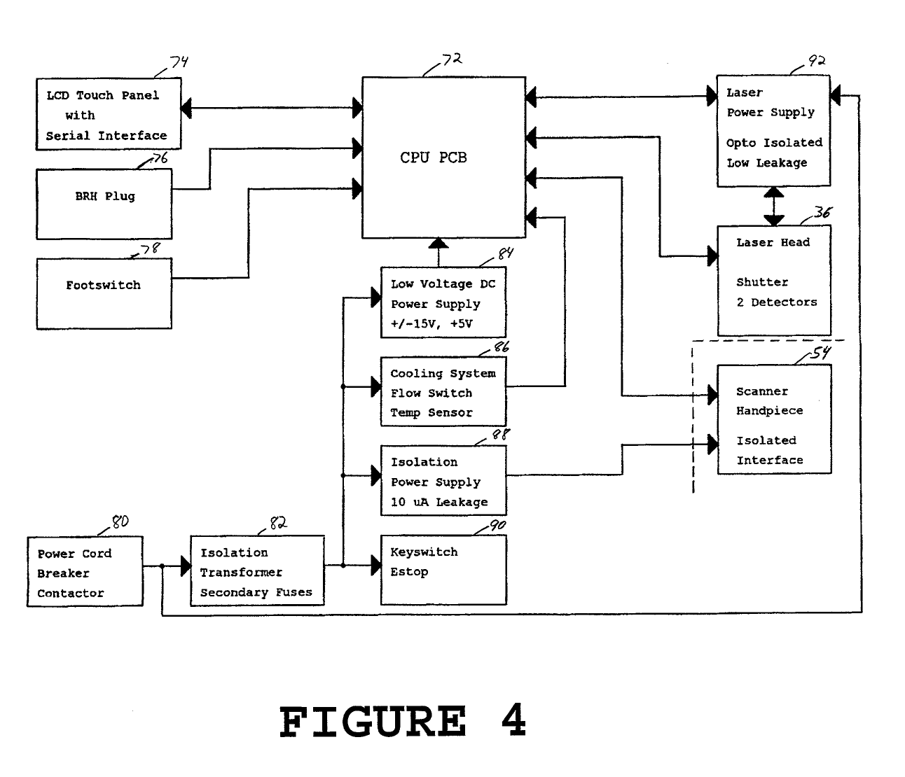

A block diagram of the electrical components and connections within the laser system of the preferred embodiment of the present invention is illustrated in FIG. 4. An LCD touch panel 74 is coupled to a central processing unit (CPU) 72. The LCD touch panel 74 provides a graphical user interface to the user to provide communications to the user and receive input commands from the user for operation of the laser system. Through this LCD panel 74 the user is provided with a display of current settings and has the ability to change settings by touching appropriate locations on the touch panel. As will be apparent to those skilled in the art, any other appropriate display and input device could alternatively be used within the laser system of the present invention. A footswitch 78 is also coupled to the CPU 72 and is used by the user to control operation of the laser system in a known manner. A safety interlock plug 76 is coupled to the CPU to allow for connection of a door or other interlock to the system. If the interlock is broken the laser is disabled. A power cord 80 is coupled to provide power to the laser system of the present invention. The power cord 80 is coupled to an isolation transformer 82 and to a laser power supply 92 for providing power to components within the laser system. The isolation transformer 82 is coupled to provide power to a keyswitch 90, an isolation power supply 88, a cooling system 86 and a low voltage DC power supply 84. The cooling system 86 monitors the temperature within the laser system and operates in order to maintain the temperature within an acceptable operating range. The cooling system 86 is also coupled to the CPU 72. The low voltage DC power supply 84 is coupled to provide power to the CPU 72.

The laser power supply 92 is coupled to the CPU 72 and to the laser head or galvonometer 36 from which the laser output 37 is provided. Preferably, the laser power supply 92 is optically isolated from the other electrical sub-systems in order to insure patient safety and prevent patient exposure to any leakage from the high voltage laser power supply 92. The laser head 36 is also coupled to the CPU 72. The scanner handpiece 54 is coupled to receive power from the isolation power supply 88. The scanner handpiece 54 is also coupled to the CPU 72.

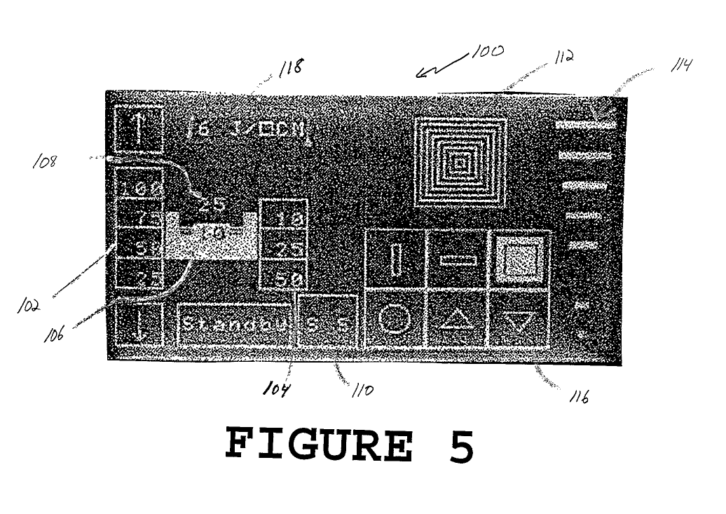

A graphical user interface of the preferred embodiment of the present invention is illustrated in FIG. 5. The graphical user interface is provided on the LCD touch panel 74. The graphical user interface 10 includes an ablation depth control section 102 in which the user selects the desired depth of ablation. Once the desired depth of ablation is selected, the selected ablation depth is displayed in the ablation depth display area 108. In the example illustrated in FIG. 5, the selected ablation depth is 25 microns. The user also selects the desired depth of coagulation from the coagulation depth control section 104. Once the desired depth of coagulation is selected, the selected coagulation depth is displayed in the coagulation depth display area 106. The coagulation depth of 10 microns is the thinnest depth of coagulation that can be selected because this is the depth of the coagulation region which will naturally occur from the delivery of an ablation pulse from an erbium laser. In the example illustrated in FIG. 5, the selected coagulation depth is 10 microns. Once the user has selected the desired depths of ablation and coagulation, the CPU 72 determines the appropriate fluence to be used. This fluence is displayed in the fluence display area 118. In the example illustrated in FIG. 5, the appropriate fluence is 6 Joules/cm².

The mode selection button 110 is used to select between a single shot or manual mode and a scanning mode. In the example illustrated in FIG. 5, the laser system is in the scanning mode. In order to switch to the single shot mode, the user presses the mode selection button 110. When in the scanning mode, the mode selection button 110 preferably includes the designation S_S to signify that by pressing the mode selection button 110, the user will cause the system to switch to the single shot mode. When in the single shot mode, the mode selection button 110 preferably includes the designation PAT to signify that by pressing the mode selection button 110, the user will cause the system to switch to the scanning mode.

When in the scanning mode, the user can select the pattern and scan size by which the laser will be delivered to the area to be treated. The pattern selection section 116 includes representations of a number of patterns which the user can select by pressing the corresponding area within the graphical user interface 100. The selected pattern is highlighted within the graphical user interface 100. In the example illustrated in FIG. 5, a square pattern has been selected. The scan size selection section 114 includes a number of square size selections from which the user can select the desired scan size by pressing the corresponding area within the graphical user interface 100. A pattern representation 112 of the selected pattern and the selected scan size is displayed within the graphical user interface 100.

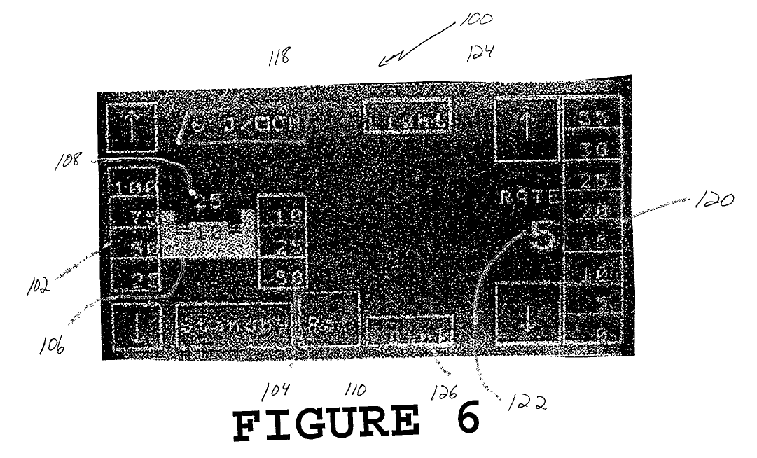

An example of the graphical user interface 100 within the single shot mode is illustrated in FIG. 6. Within this mode the graphical user interface 100 includes a rate selection section 120 in which the user selects the rate at which the laser pulses are delivered. Once the desired rate is selected, the selected rate is displayed in the rate display area 122. In the example illustrated in FIG. 6, the selected rate is 5 pulses per second. The graphical user interface also includes display intensity control buttons 124 and 126 by which the user can control the intensity of the display within the graphical user interface 100.

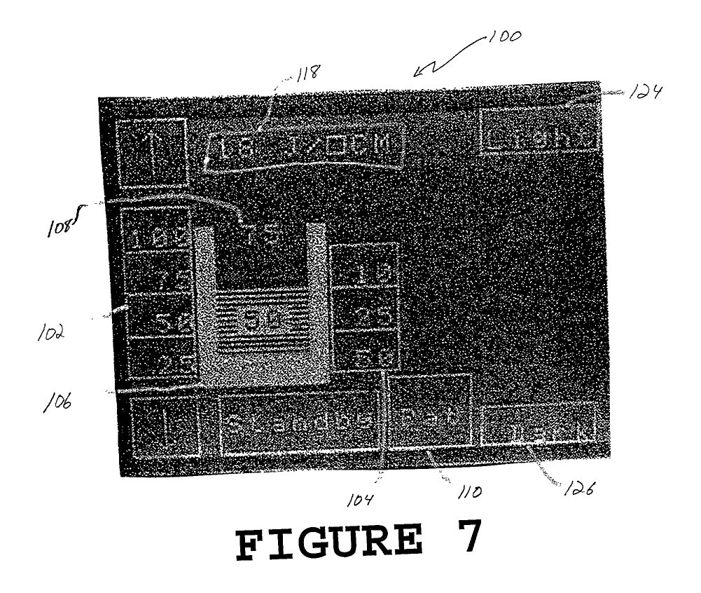

An enlarged example of the left side of the graphical user interface 100 of the present invention is illustrated in FIG. 7. In the example of FIG. 7, the selected ablation depth is 75 microns and the selected coagulation depth is 50 microns. These depths are displayed in numeric form. As shown in FIG. 7, the graphical user interface also includes a graphical representation showing the size of the ablation well highlighted around the numeric display of the selected ablation depth 108 and the selected coagulation depth 106 which corresponds to the selected depths. The appropriate fluence corresponding to the selected depths in the example of FIG. 7 is 18 Joules/cm².

As described above, the laser system of the present invention operates in two modes. The ablation mode combines a series of one or more pulses from the laser source 31 delivered to the skin area 58 in order to ablate the skin area to be treated to the ablation depth selected by the user. Within this mode, ablation of any desired depth of skin can be achieved in order to create a crater of a desired size at the skin area 58. As described above, the final pulse within this series of ablation pulses will result in a coagulation zone having a thickness of 10 microns, as illustrated in the crater 22 of FIG. 2. However, it is believed that a greater depth of coagulation will promote a stronger healing response within the treated area of skin. Using the coagulation mode of the laser system of the present invention, the coagulation zone at the skin area 58 can be increased to a desired depth below the remaining exposed layer of skin.

When in the coagulation mode, the laser system of the present invention, provides a series of non-ablative pulses to the surface of the remaining exposed layer in order to raise the temperature at the surface of the skin. The heat at the surface of the skin created by the coagulation pulses is then conducted from the surface of the skin into a depth of the skin, thereby raising the temperature of the depth of skin below the surface and creating a coagulation region. By controlling the energy of the non-ablative coagulation pulses and the time which the surface of the skin is exposed to these pulses, the depth of the coagulation zone below the remaining exposed layer of skin can be controlled.

When a coagulation depth thicker than 10 microns is selected by the user, the laser system of the present invention will first provide the ablation pulses to the area to be treated in order to ablate the patient's skin to the selected ablation depth. The laser system then provides the non-ablative coagulation pulses to the treated area in order to generate a coagulation region having the selected depth. The coagulation pulses raise the temperature at the surface of the skin for a predetermined period of time. This causes the temperature below the surface of the skin to also rise which creates a coagulation skin under the surface of the skin. By controlling the length of time the skin surface is maintained at an elevated temperature, the depth of coagulation under the surface of the skin can be controlled.

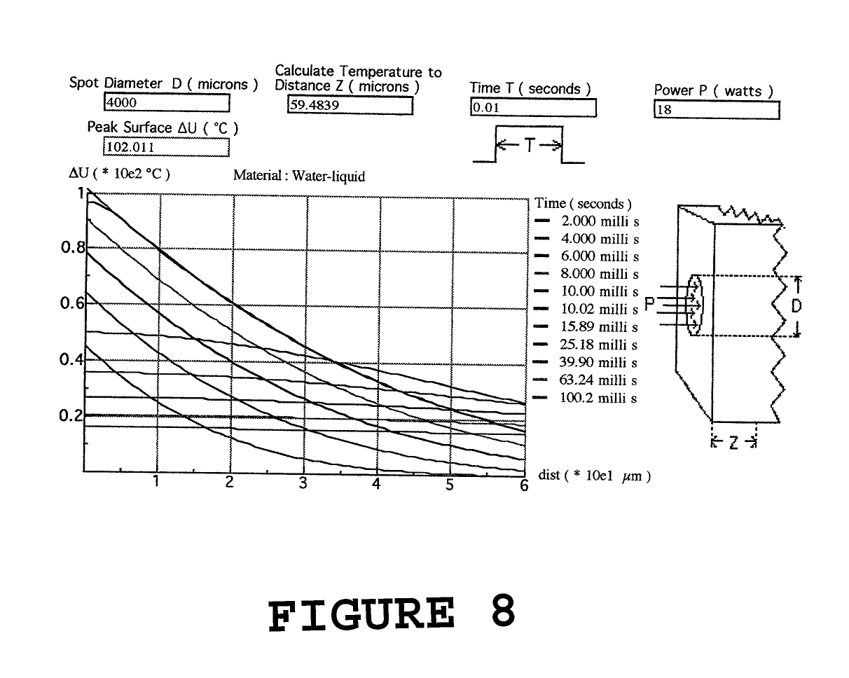

A graph illustrating the effect of the coagulation pulses of the present invention is illustrated in FIG. 8. In the example illustrated in FIG. 8, the coagulation pulses are provided to a spot having a diameter of 4000 microns over a time period of 10 milliseconds at a fluence of 18 Joules/cm in order to generate a coagulation region having a depth of 50 microns below the surface of the skin. The graphs in FIG. 8 illustrate the temperature versus depth under the surface of the skin at specified time periods. The non-ablative coagulation pulses raise the temperature at the surface of the skin within the spot to 102 degrees Celsius. Over the time periods shown the temperature is raised by 30 degrees Celsius at a depth of 50 microns. Since the skin is initially near the normal body temperature of 37 degrees Celsius, raising the temperature of skin by 30 degrees Celsius will result in a temperature of 67 degrees, sufficient to cause coagulation and generate the coagulation region to the desired depth of 50 microns.

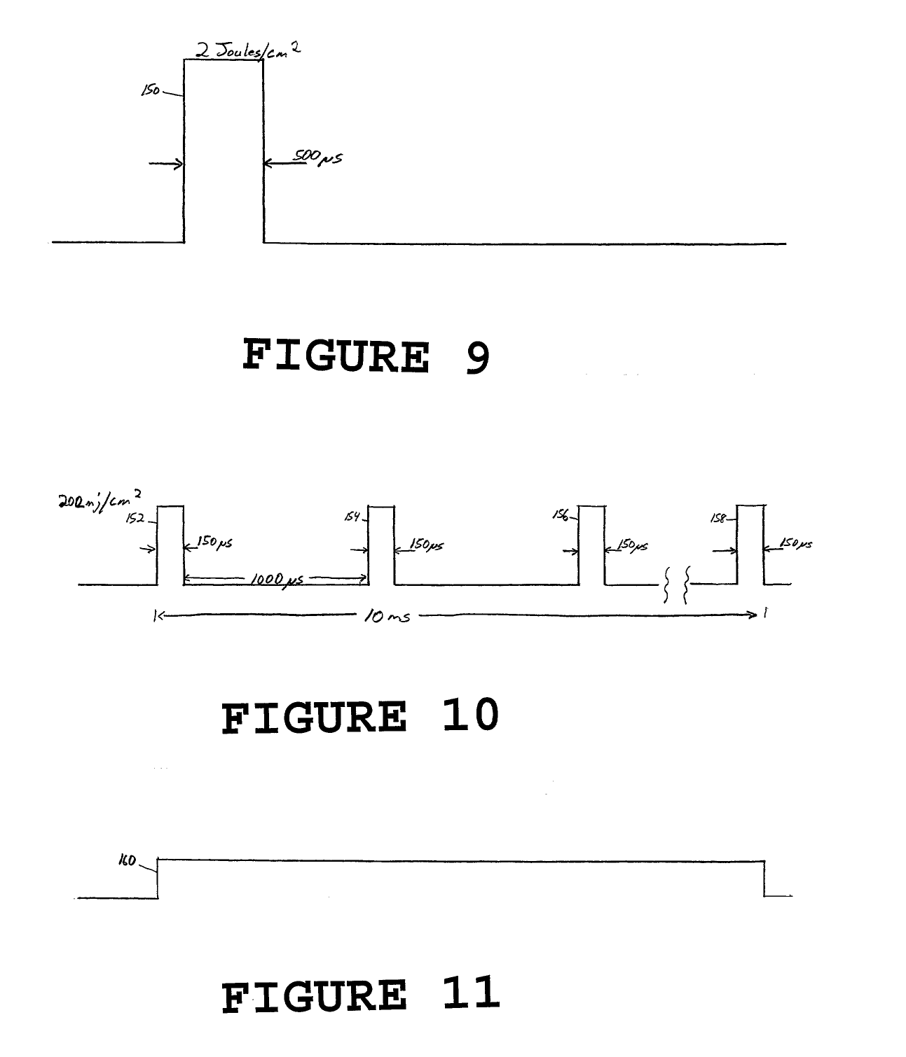

An ablation pulse delivered from the laser system of the present invention is illustrated in FIG. 9. The ablation pulse 150 illustrated in FIG. 9 has a fluence of 2 Joules/cm² and a duration of 500 microseconds. The ablation pulse 150 will ablate a depth of skin at the area of skin to which the pulse is delivered. As is well known, the duration or fluence of the pulse can be adjusted or a combination of ablation pulses can be delivered in order to achieve the desired depth of ablation.

A coagulation pulse sequence delivered from the laser system of the present invention is illustrated in FIG. 10. The coagulation pulses 152, 154, 156 and 158 each have a fluence of 200 milliJoules/cm² and a duration of 150 microseconds. The coagulation pulses are delivered every millisecond over a time period of 10 milliseconds. The coagulation pulses are of a fluence and duration which will not ablate the skin. However, the series of coagulation pulses will raise the temperature at the surface of the skin, causing the temperature below the skin to rise above the coagulation temperature threshold to the desired depth, thereby resulting in the appropriate depth of the resulting coagulation region.

The laser system of the present invention preferably delivers a sequence of coagulation pulses over a period of time at periodic intervals in order to generate a coagulation region having the selected depth. Alternatively, a single pulse 160 of an appropriate fluence and duration, as illustrated in FIG. 11, is used to raise the temperature at the surface of the area of skin to be treated in order to achieve a coagulation region having the desired depth. However, conventional lasers operate inefficiently at lower fluence levels, so the method of FIG. 10 is presently preferred.

In operation, a user selects the appropriate settings for the desired treatment using the laser system and the graphical user interface of the present invention. Using the touch panel and the graphical user interface, the user selects the ablation depth, the coagulation depth and if in the scanning mode, the scan pattern and scan size. If the user has selected the single shot mode, then the user must select the rate at which the laser pulses will be generated. Once the user has made the appropriate selections using the graphical user interface 100 and the touch panel 74, the CPU 72 then selects the appropriate fluence to achieve the desired ablation and coagulation. The user then positions the scanner hand piece 54 at the surface of the patient's skin to be treated. Once the scanner hand piece 54 is in the correct position, the user then toggles the footswitch 78 and the ablation pulse or pulses are generated from the laser source 31, delivered through the arm 38 and scanner handpiece 54 to the spot of the patient's skin being treated in order to ablate the selected depth of skin at the spot. Once the ablation pulses have been delivered and the skin area has been ablated to the selected depth, the coagulation pulses are generated by the laser source 31, delivered through the arm 38 and scanner handpiece 54 to the spot of the patient's skin being treated in order to generate the selected depth of coagulation. The coagulation pulse sequence is only generated if the user has selected a coagulation depth greater than 10 microns. If the user has selected a coagulation depth of 10 microns, then the ablation pulses will generate this depth of coagulation as described above, without the need for a separate sequence of coagulation pulses.

By providing a single laser which as the ability to both ablate an area of skin to a selected depth and generate a coagulation region to a selected depth, it is not necessary for a user to have multiple lasers for the performance of a wide range of skin resurfacing procedures. With the single laser system of the present invention, the user has the flexibility to treat and resurface skin damaged due to prolonged exposure to the sun and blemishes such as wrinkles with a single laser system. The user also has the ability to control the ablation depth and the coagulation depth in order to specifically tailor the treatment to the condition of the patient's skin and the best treatment which will ablate the skin to the appropriate depth and generate a coagulation region of the depth necessary to promote the best healing response.

In the preferred embodiment of the present invention, an erbium laser is used. Alternatively, any appropriate short penetration laser source can be used within the system of the present invention.

As discussed above, the laser system of the present invention includes the articulated arm 38 to deliver the laser from the laser head 36 to the scanner handpiece 54. Within the arm 38 are a series of focussing lenses 46, 50 and 52 which are utilized to refocus the laser beam 37 as it travels through the arm 38. As is well known in the art, a laser beam travelling over a distance will converge until it reaches its focal point and then will tend to naturally expand as it travels past its focal point. The focussing lenses 46, 50 and 52 refocus the laser beam 37 so that the laser beam delivered to the scanner handpiece 54 is the same diameter as the laser beam output from the laser source 31. Previous medical laser systems have accounted for the natural expansion of the laser beam over a distance by delivering a small laser beam from the laser source so that when it reaches the scanner handpiece it is the appropriate size. However, this requires that the arm be constructed to strict mechanical tolerances so that its length is precisely known. By including the relay focussing lenses of the present invention within the articulated arm 38, the appropriate size laser beam 37 can be delivered from the laser source 31 as is required at the delivery point, thereby increasing the strength of the laser beam which can be delivered. The mechanical tolerance requirements of the delivery system are also greatly diminished due to the inclusion of the relay focussing lenses within the articulated arm.

Preferably, the focussing lenses 46, 50 and 52 are simple convex lenses. Alternatively, any other appropriate lenses can be used.

The present invention has been described in terms of specific embodiments incorporating details to facilitate the understanding of principles of construction and operation of the invention. Such reference herein to specific embodiments and details thereof is not intended to limit the scope of the claims appended hereto. It will be apparent to those skilled in the art that modifications may be made in the embodiment chosen for illustration without departing from the spirit and scope of the invention.

We claim:

1. A medical laser delivery apparatus for delivering one or more pulses to an area of tissue to be treated and generating a region of coagulation to a controllable coagulation depth under a surface of the area of tissue comprising a laser source for generating a series of one or more non-ablative pulses to be delivered to the area of tissue to be treated in order to raise a temperature at the surface of the area of tissue to be treated to a temperature sufficient to generate coagulation at the coagulation depth when the laser source is in a coagulation mode.

2. The medical laser delivery apparatus as claimed in claim 1 further comprising a laser delivery system coupled to the laser source for delivering the one or more pulses from the laser source to the area of tissue to be treated.

3. The medical laser delivery apparatus as claimed in claim 2 wherein the laser delivery system comprises an articulated arm and one or more refocussing optics for refocussing the laser pulses as they travel through the arm.

4. The medical laser delivery apparatus as claimed in claim 3 wherein the laser delivery system further comprises a scanning handpiece at an end of the arm for providing the laser pulses to the area of tissue being treated.

5. The medical laser delivery apparatus as claimed in claim 4 wherein the refocussing optics are simple convex lenses.

6. The medical laser delivery apparatus as claimed in claim 1 further comprising a graphical user interface through which a user selects the coagulation depth and/or fluence.

7. The medical laser delivery apparatus as claimed in claim 6 wherein the laser source also has an ablation mode wherein it generates laser pulses of a strength and duration to ablate tissue at the area of tissue being treated to an ablation depth and the user selects the ablation depth through the graphical user interface.

8. The medical laser delivery apparatus as claimed in claim 1 wherein the laser source includes a laser having a short penetration depth.

9. The medical laser delivery apparatus as claimed in claim 8 wherein the laser is an erbium laser.

10. The medical laser delivery apparatus as claimed in claim 8 wherein the laser is an Er:YAG laser.

11. A medical laser comprising:
a. a laser source for generating a laser beam having a predetermined absorption length, wherein the absorption length forms a predetermined coagulation depth in response to an ablative laser pulse; and
b. a laser control system, coupled for controlling the laser source for generating a plurality of coagulative laser pulses, such that each such coagulative laser pulse is delivered in sequence to a target area to form a coagulation region deeper than the predetermined coagulation depth.

12. The medical laser as claimed in claim 11 further comprising a graphical user interface through which a user selects a depth of the coagulation region formed by the coagulative laser pulses.

13. The medical laser as claimed in claim 12 further comprising a laser delivery system coupled to the laser source for delivering the laser beam from the laser source to an area of tissue to be treated.

14. The medical laser as claimed in claim 13 wherein the laser delivery system comprises an articulated arm and one or more refocussing optics for refocussing the laser beam as it travels through the arm.

15. A method of delivering laser pulses to an area of tissue to be treated and generating coagulation to a controllable coagulation depth under a surface of the tissue comprising the steps of:
a. generating a series of one or more non-ablative pulses from a laser source;
b. delivering the series of one or more non-ablative pulses to the area of tissue to be treated in order to raise the tissue to be treated to a temperature sufficient to generate coagulation at the coagulation depth.

16. The method of delivering laser pulses as claimed in claim 15 further comprising the step of displaying a graphical user interface through which a user selects the coagulation depth.

17. A medical laser delivery apparatus for treating an area of tissue comprising:
a. a laser source for generating a series of one or more laser pulses each having a strength and a duration;
b. a laser delivery system coupled to the laser source for delivering the laser pulses from the laser source to the area of tissue being treated;
c. a control system coupled to the laser source for controlling generation of the laser pulses from the laser source, wherein the laser source operates in both an ablation mode and a coagulation mode such that when in the ablation mode, the strength and duration of the laser pulses are sufficient to ablate tissue at the area of tissue being treated to a controllable ablation depth and when in the coagulation mode, the strength and duration of the laser pulses are sufficient to generate a coagulation region having a controllable coagulation depth within the tissue remaining at the area of tissue being treated without ablating any tissue.

18. The medical laser delivery apparatus as claimed in claim 17 further comprising a graphical user interface through which a user selects the controllable ablation depth and the controllable coagulation depth.

19. The medical laser delivery apparatus as claimed in claim 18 wherein the laser delivery system comprises an articulated arm and one or more refocussing optics for refocussing the laser beam as its travels through the articulated arm.

20. The medical laser delivery apparatus as claimed in claim 19 wherein the laser delivery system further comprises a scanning handpiece at an end of the arm for providing the laser pulses to the area of tissue being treated.

21. The medical laser delivery apparatus as claimed in claim 20 wherein the refocussing optics are simple convex lenses.

22. The medical laser delivery apparatus as claimed in claim 21 wherein the laser source includes a laser having a short penetration depth.

23. The medical laser delivery apparatus as claimed in claim 22 wherein the laser is an erbium laser.

24. The medical laser delivery apparatus as claimed in claim 22 wherein the laser is an Er:YAG laser.

25. A graphical user interface for monitoring and controlling operation of a medical laser system in the treatment of an area of tissue comprising:
a. an ablation control section through which a user selects a desired ablation depth specifying how much tissue is to be ablated at the area of tissue being treated;
b. a coagulation control section through which a user selects a desired coagulation depth specifying how thick a coagulation region is to be generated at the area of tissue being treated; and
c. a representation of the area of tissue being treated illustrating the selected ablation depth and the selected coagulation depth.

26. The graphical user interface as claimed in claim 25 wherein the representation of the area of tissue being treated displays the selected ablation depth and the selected coagulation depth in numeric form and represents the selected ablation depth and the selected coagulation depth in a graphical form.

27. The graphical user interface as claimed in claim 26 wherein the graphical user interface operates in a scanning mode and a non-scanning mode.

28. The graphical user interface as claimed in claim 27 further comprising a scan pattern control section which is displayed when the graphical user interface is in the scanning mode and through which the user selects a desired scan pattern and scan size.

29. The graphical user interface as claimed in claim 28 further comprising a rate selection control section which is displayed when the graphical user interface is in the non-scanning mode and through which the user selects the rate at which laser pulses are to be delivered to the area of tissue being treated.

30. A method of monitoring and controlling a medical laser system in the treatment of an area of tissue comprising the steps of:
a. displaying a plurality of ablation depths from which a desired ablation depth is selected, wherein the ablation depth specifies how much tissue is to be ablated at the area of tissue being treated;
b. displaying a plurality of coagulation depths from which a desired coagulation depth is selected, wherein the coagulation depth specifies how thick of a coagulation region is to be generated at the area of tissue being treated; and
c. displaying a representation of the area of tissue being treated illustrating the selected ablation depth and the selected coagulation depth.

31. The method as claimed in claim 30 wherein the representation of the area of tissue being treated displays the selected ablation depth and the selected coagulation depth in numeric form and represents the selected ablation depth and the selected coagulation depth in a relative graphical form.

32. The method as claimed in claim 31 further comprising the step of displaying a plurality of scan patterns and scan sizes from which a desired scan pattern and a desired scan size are selected.

33. The method as claimed in claim 32 further comprising the step of displaying a plurality of rates of delivery of laser pulses from which a desired rate of delivery is selected.

34. A medical laser delivery system for delivering a laser beam from a laser source to an area of tissue to be treated by the laser beam comprising one or more focussing optics for refocusing the laser beam as it travels through the delivery system.

35. The medical laser delivery system as claimed in claim 34 further comprising an articulated arm in which the focussing optics are mounted and through which the laser beam travels.

36. The medical laser delivery system as claimed in claim 35 wherein the articulated arm includes one or more directing optics for directing the laser beam from the laser source through the articulated arm to the area of tissue to be treated.

37. The medical laser delivery system as claimed in claim 36 wherein the focussing optics are simple convex lenses.

38. An articulated arm laser delivery system for delivering a laser beam from a laser source to an area of tissue to be treated by the laser beam comprising:
a. a first arm component;
b. a second arm component;
c. a joint coupling the first arm component and the second arm component;
d. a plurality of directing optics for directing the laser beam from the laser source through the first arm, the joint and the second arm to the area of tissue to be treated; and
e. one or more focussing optics for refocussing the laser as it travels through the first and second arms.

39. The articulated arm laser delivery system as claimed in claim 38 further comprising a scanning handpiece mounted on an end of the second arm component and through which the laser beam is delivered to the area of tissue to be treated.

40. The articulated arm laser delivery system as claimed in claim 39 wherein the focussing optics are simple convex optics.
